(19) United States
(12) Patent Application Publication
(10) Pub. No.: US 2025/0369354 A1
(43) Pub. Date: Dec. 4, 2025

(54) METHOD AND SYSTEM FOR MEASURING RHEOLOGY OF DRILLING FLUID USING MICROCHIPS AND MODIFIED POISEUILLE'S FORMULA

(71) Applicant: SAUDI ARABIAN OIL COMPANY, Dhahran (SA)

(72) Inventors: Mohammed ALABBAD, Dhahran (SA); Naif J. ALQAHTANI, Dhahran (SA); Abrar A. ALSHAIKH, Dhahran (SA); Bodong LI, Dhahran (SA)

(73) Assignee: SAUDI ARABIAN OIL COMPANY, Dhahran (SA)

(21) Appl. No.: 18/731,173

(22) Filed: May 31, 2024

(51) Int. Cl. E21B 49/00; E21B 47/07; G01N 11/00; G01N 33/28

(52) U.S. Cl. CPC E21B 49/005; E21B 47/07; G01N 11/00; G01N 33/2823

(57) ABSTRACT

System and methods are disclosed relating to the measuring of the rheological properties of drilling fluid used in a borehole with an annulus by using microchip sensors configured to traverse the annulus along with the drilling fluid to measure pressure, temperature, and various other parameters as functions of depth or time, and applying a modified version of Poiseuille's formula to said measurements to map the rheological properties of the drilling fluid.

### FIELD OF THE DISCLOSURE

This disclosure relates generally to measuring rheology of drilling fluid, and more specifically, to utilizing microchip sensors and a modified Poiseuille's formula to map viscosity and measure the rheology of drilling fluid.

### BACKGROUND OF THE DISCLOSURE

Drilling fluids, or drilling muds, are crucial elements of any drilling process or project. Drilling fluid is typically a viscous fluid mixture that is pumped down the hollow drill string to the drill bit, where it exits and is flushed back up the annulus to the surface. It serves many functions, such as controlling formation pressures, removing cuttings from the borehole, sealing permeable formations during drilling, cooling and lubricating the drill bit, and stabilizing the borehole through hydrostatic pressure. Drilling fluids come in many compositions, each being designed to meet specific borehole demands, such as controlling subsurface pressures, minimizing formation damage, controlling erosion, and optimizing hole cleaning.

Viscosity is a major physical property of fluid that describes its resistance to flow. For drilling mud, it can range from high-viscosity “thick” to low-viscosity “thin”. Funnel viscosity and plastic viscosity are the two main techniques used in the industry to determine mud viscosity at the surface due to their simplicity and quickness. However, these techniques do not represent the true viscosity downhole due to fluctuations in temperature, pressure, shear rate, and time delay between measurements. Moreover, these viscosity tests are usually conducted manually and thus cannot provide continuous viscosity measurements. Knowing the viscosity of the drilling fluid throughout its circulation through the borehole is a critical expedient that will provide more holistic measurements of downhole parameters, determine inflow/outflow sections, and assist drilling engineers in adjusting drilling fluid parameters for better well control, cleaning, and meeting the varying demands of unique wells.

### SUMMARY OF THE DISCLOSURE

Various details of the present disclosure are hereinafter summarized to provide a basic understanding. This summary is not an extensive overview of the disclosure and is neither intended to identify certain elements of the disclosure nor to delineate the scope thereof. Rather, the primary purpose of this summary is to present some concepts of the disclosure in a simplified form prior to the more detailed description that is presented hereinafter.

According to an embodiment consistent with the present disclosure, a system for measuring the rheology of drilling fluid used in a borehole having an annulus includes one or more microchip sensors configured to traverse at least a portion of the annulus along with the drilling fluid to thereby measure pressure and temperature as functions of depth or time in said portion, and a computing system comprising a processor and a memory and a viscosity mapper, the viscosity mapper operable to receive said pressure and temperature measurements and apply a modified Poiseuille's formula to determine a mapping of rheological properties of the drilling fluid.

According to another embodiment, a method for measuring drilling fluid rheology of an annulus of a borehole includes delivering one or more microchip sensors along with the drilling fluid into the annulus, obtaining from the delivered microchip sensors measurements of pressure and temperature as functions of depth or time in the annulus, and applying a modified Poiseuille's formula to said measurements to determine a mapping of rheological properties of the drilling fluid.

In a further embodiment, a machine-readable storage medium having stored thereon a computer program for measuring drilling fluid rheology of an annulus of a borehole is disclosed. The computer program includes a routine of set instructions for causing the machine to perform the steps of delivering one or more microchip sensors along with the drilling fluid into the annulus, obtaining from the delivered microchip sensors measurements of pressure and temperature as functions of depth or time in the annulus, and applying a modified Poiseuille's formula to said measurements to determine a mapping of rheological properties of the drilling fluid.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a block diagram of an example of a system for measuring the rheology of drilling fluid used in a borehole having an annulus.

FIG. 2 is block diagram depicting an example of a rheology analyzer.

FIG. 3 is a diagram depicting an example schematic of an annulus from which the modified Poiseuille's formula may apply to in accordance with certain embodiments.

FIG. 4 is a diagram depicting an example of a mapping of rheological properties, according to one embodiment.

FIG. 5 is a is flowchart diagram depicting an example of a method for measuring drilling fluid rheology of an annulus of a borehole.

FIG. 6 is a block diagram of a computer system that may be used to implement one or more of the systems or methods described herein in accordance with certain embodiments.

FIG. 7 depicts a cloud computing environment that can be used to perform one or more actions according to an aspect of the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments of the present disclosure relate to measuring rheology of drilling fluid, and more specifically, to utilizing microchip sensors and a modified Poiseuille's formula to map viscosity and measure the rheology of drilling fluid.

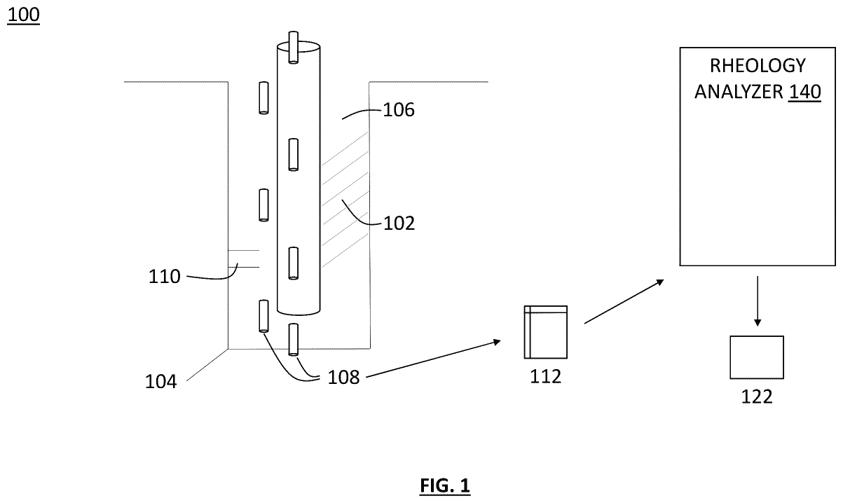

FIG. 1 is a block diagram of a system 100 for measuring the rheology of drilling fluid 102 used in a borehole 104 having an annulus 106 in accordance with certain embodiments.

A drilling fluid may be any fluid or gas mixture of fluids and/or gases used to aid in the drilling of boreholes into the earth. A drilling fluid, often also referred to as a drilling mud, can be composed of natural and/or synthetic material in a mixed state. For example, two common types of drilling fluids are water-based muds (WBs) and non-aqueous muds, often called oil-based muds (OBs). Drilling fluids may also be gases, such as air or mist, in select situations. Drilling fluids may serve many functions including, but not limited to, exerting pressure to prevent formation fluids from entering the well, removing drill cuttings, suspending drill cuttings and weight material, cooling and lubricating a drill bit and drill string, and adding buoyancy to the drill string. A borehole can be any narrow shaft bored into the ground. A borehole can be created for many purposes, such as the extraction of water, other liquids like petroleum, or gases such as natural gas. It may also be used for mineral exploration, temperature measurement, geothermal installations, underground storage, environmental assessment, or any geotechnical investigation. An annulus is any region, space, or area between two concentric circles—or in three dimensions—between two concentric cylinders. In drilling, an annulus of a drill well may be any void between piping, tubing, or casing and the piping, tubing, or casing surrounding it. For example, in a new well in the process of being drilled, an annulus may be the void between the drill string and the surrounding formation being drilled. An annulus may allow for circulating fluid in the well by pumping a drilling fluid down the inside of the drill string and then up through the annulus to the surface, or vice versa. In another embodiment, there may be more than one annuli, such as in a completed well.

System 100 includes one or more microchip sensors 108 configured to traverse at least a portion 110 of the annulus 106 along with the drilling fluid 102 to thereby measure pressure and temperature as functions of depth and/or time in said portion 110. A portion of the annulus may be any distinct section or cross section of the annulus defined by any two or three dimensional area. For example, a portion of the annulus may be a rectangular, cylindrical, or spherical area. These areas may be of any interest in a borehole for the measurement of parameters. For example, there may be an area near a particular subterranean formation where measurements must be taken to assess flow or viscosity and thus adjust the annulus flow accordingly. Similarly, a portion may also include the entire depth profile of the annulus, to measure a more holistic picture of the parameters and thus the rheological properties of the fluid across the entire annulus.

A microchip sensor 108 may be any miniaturized electronic system configured to detect certain parameters. Such parameters a microchip sensor may measure include but are not limited to pressure, temperature, location, distance between different microchip sensors, flow path, volume, density, drilling fluid flow rate, equivalent shear, stress, strain, pH, and annulus radius. In embodiments, the microchip sensors 108 may measure parameters within the borehole 104, within the drilling fluid 102, within the surrounding earth formation, or any combinations thereof. Microchip sensors 108 may be deployed into the borehole 104 and/or annulus 102 via any means, including but not limited to, through the drill string, through the annulus 102, or pre-positioned locally within portions 110 of the bore hole. The sensors may traverse the annulus 102 in static or dynamic flow paths, circulated in forward or reverse trajectories, at static or dynamic speeds, and/or maintain static positions. The varying locations and trajectories of the microchip sensors will allow for the measuring of the pressure and/or temperature, as well as other parameters, from the surface to the bottom of the drill string or a bottom-hole assembly through the drill string and then back to the surface through the annulus 102, or vice versa using reverse circulation or some mechanical release or recovery tools. In embodiments, the microchip sensors 108 can traverse and measure parameters before, after, or during the active process of drilling. Recovery of said microchip sensors may also occur during some or all of these phases. Such a continuous deployment and recovery of the microchip sensors may provide a semi-continuous data stream. In another embodiment, the microchip sensors 108 may comprise at least one magnetometer to detect the position of each of the microchip sensors against the depth of the annulus. The microchip sensors may also comprise at least one accelerometer to detect the position of the microchip sensors against the depth of the annulus. In some embodiments, the microchip sensors may include a recovery mechanism for receiving the said pressure and/or temperature measurements or other parameters from the microchip sensors for delivery to the viscosity mapper discussed below.

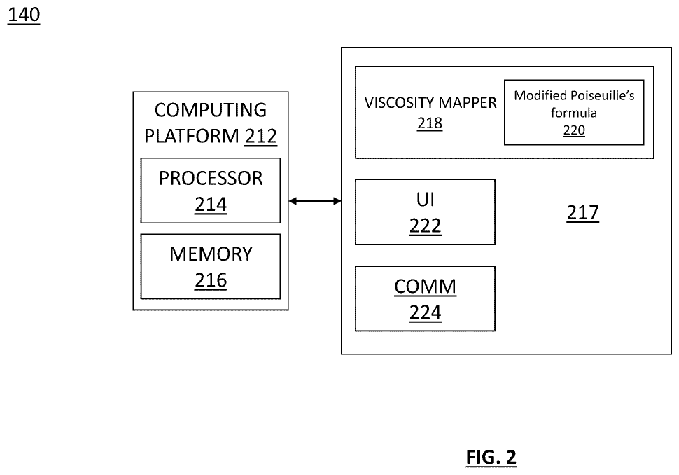

Also included in the system is a rheology analyzer 140 in operative communication with the microchip sensors 108 and shown in more detail in FIG. 2. Rheology analyzer 140 comprises a computing platform 212 in which a processor 214 executes instructions that may be stored in a memory 216 for implementing a viscosity analysis module 217. The viscosity analysis module 217 may also include a user interface 222, operable to provide an interactable medium to a user, and a communications module 224, operable to send and receive information between internal modules or any external communicable devices, such as the microchip sensors 108. The viscosity analysis module 217 can include a viscosity mapper 218, wherein the viscosity mapper is operable to receive said pressure and/or temperature measurements from microchip sensors 108 and apply a modified Poiseuille's formula 120 to determine a mapping 122 (FIG. 1) of rheological properties of the drilling fluid 102.

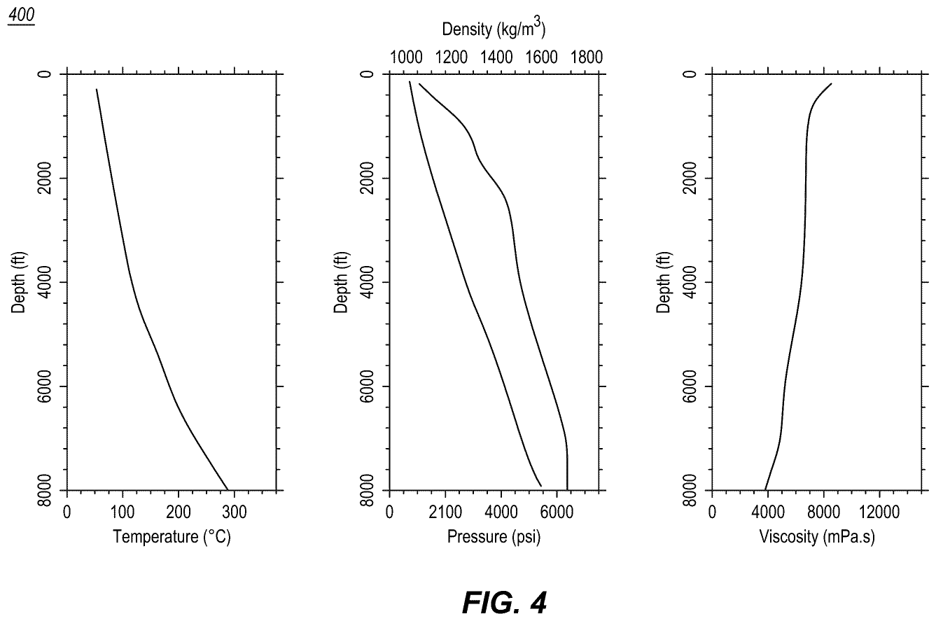

Poiseuille's formula is a physical law that provides the change in pressure in a laminar flow flowing through a cylindrical pipe. The standard formula, solved for viscosity η, is provided by:

$$η = \frac{Δ P π {r}^{4}}{8 Q L}$$

where η is the viscosity of the fluid, ΔP is the pressure gradient, r is the radius of the cylinder, L is the length of the cylinder, and Q is the flow rate. The flow of the fluid is found to vary inversely with the viscosity of fluid. For example, an increase in the viscosity of the fluid will result in a decrease of the flow, and similarly the flow will increase as viscosity increases. The difference in pressure which exists between the two ends of the cylinder is determined by the fact that fluid will always flow from high pressure regions to low pressure regions. For example, the flow rate will be determined by the pressure gradient of (P1-P2). The flow of fluid varies directly with the radius to a power of 4. For example, if the radius is halved then the flow decreases by 16 times. The flow is inversely proportional to the cylinder length and thus the longer the cylinder length the greater is the resistance to the flow. Poiseuille's formula may be used to determine drilling fluid viscosity in the drill string. However, and with reference to FIG. 4, a modified Poiseuille's formula must be derived and utilized to determine drilling fluid viscosity in the annulus 106, using measurements provided by the microchip sensors 108. Such a modified Poiseuille's formula may be provided by:

$$𝒬 = \frac{π ( {r}_{2}^{2} - {r}_{1}^{2} )}{8 μ} ( - \frac{\partial p}{\partial z} ) [ {r}_{2}^{2} + {r}_{1}^{2} - \frac{{r}_{2}^{2} - {r}_{1}^{2}}{ln ( {r}_{2} / {r}_{1} )} ]$$

where μ is the viscosity of the fluid, r₁ is the radius of the inner cylinder, and r₂ is the radius of the outer cylinder. In other respects, such as the remaining variables, the same definitions as described above in the context of the standard formula are maintained. The pressure gradient, in both formulas, may be determined using one or more of the microchip sensors 108 that can be deployed as one or more patches at calculated times and the drilling fluid pump rate in order to get the pressure difference with respect to the depth. The pressure measurements may be determined as a time series using a pressure sensor imbedded within the microchip sensors, while the depth can be estimated from the flowrate and the time interval it takes from deployment to recovery of the microchip sensor, or through other means.

Viscosity mapper 218 may be any dedicated computing module capable of receiving temperature and pressure measurements from the microchip sensors 108 and then applying a modified Poiseuille's formula 120 to calculate rheological properties 122 of the drilling fluid. In some embodiments, the viscosity mapper may be operable to receive additional parameters from the microchip sensors 108, including but not limited to location, distance between different microchip sensors, flow path, volume, density, drilling fluid flow rate, equivalent shear, stress, strain, pH, and annulus radius. Additionally, the viscosity may also be operable to receive relevant parameters from other external devices that are not the microchip sensors. For example, the viscosity mapper may receive parameters such as equivalent shear rates, pumping flow rates, or rotations per minute from the drilling rig or any communicable computing module coupled thereto. In some embodiments, all these parameters, in addition to temperature and pressure, may be used to map the rheological properties of the drilling fluid 122.

A mapping of rheological properties may be any presentation of data representing the rheological properties of a drilling fluid. For example, the measured parameters such as temperature, pressure, and additional parameters described above may be analyzed by the viscosity mapper 218 using the modified Poiseuille's formula to determine a full fluid viscosity mapping from surface to bottom of the drill string and/or annulus. Viscosity mappings may include readings for the full depth of the borehole, or for discrete portions, or for any subsection in between. In another embodiment, the drilling fluid flow speed may be mapped. Mapping may include, but is not limited to, presenting the rheological properties against a depth profile of the borehole and/or annulus, against a time series, against a temperature series, or against any other known or measurable variable parameter of interest. In some embodiments, the mapping may be presented graphically, including but not limited to, in a graph, table, or chart.

FIG. 4 is a diagram depicting an example of a mapping of rheological properties, according to an embodiment. In this embodiment, three separate graphs are presented, one showing temperature in degrees Celsius mapped against depth in feet, one of pressure in psi mapped against depth in feet, and one showing viscosity in mPa·s mapped against depth. These outputs may also be provided for any time, t=x.

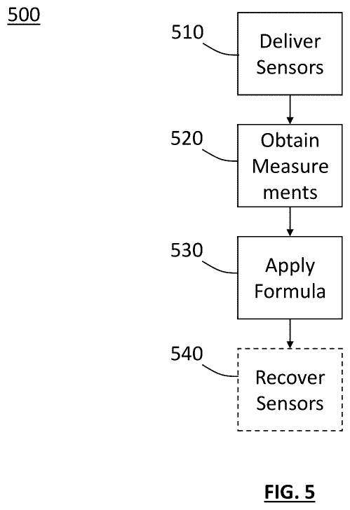

In view of the foregoing structural and functional features described above, an example(s) method will be better appreciated with reference to FIG. 5. While, for purposes of simplicity of explanation, the example method(s) of FIG. 5 is shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the method(s).

FIG. 5 is flowchart diagram depicting an example of a method 500 for measuring drilling fluid rheology of an annulus of a borehole. The method 500 can be implemented by the system 100 for measuring the rheology of drilling fluid used in a borehole having an annulus as shown in FIG. 1. Thus, reference can be made to the example of FIG. 1 in the example of FIG. 5.

Method 500 comprises delivering one or more microchip sensors along with the drilling fluid into the annulus at step 510, obtaining from the delivered microchip sensors measurements of pressure and temperature as functions of depth or time in the annulus at step 520, and applying a modified Poiseuille's formula to said measurements to determine a mapping of rheological properties of the drilling fluid at step 530.

In one embodiment, the method may 500 further include receiving one or more of drilling fluid flow rate, equivalent shear, annulus radius, and distance between different microchips to apply to the modified Poiseuille's formula.

In another embodiment, the delivering one or more microchip sensors along with the drilling fluid into the annulus at step 510 occurs continuously during an in-situ drilling process to provide a semi-continuous data stream from the annulus.

In another embodiment, method 500 may further comprise recovering the one or more microchip sensors for receiving the said pressure and temperature measurements for delivery at step 540.

In another embodiment, the modified Poiseuille's formula comprises

$$𝒬 = \frac{π ( {r}_{2}^{2} - {r}_{1}^{2} )}{8 μ} ( - \frac{\partial p}{\partial z} ) [ {r}_{2}^{2} + {r}_{1}^{2} - \frac{{r}_{2}^{2} - {r}_{1}^{2}}{ln ( {r}_{2} / {r}_{1} )} ]$$

where Q is flow rate, r₁ is annulus radius, r₂ is borehole radius, μ is viscosity of fluid, and p is pressure.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

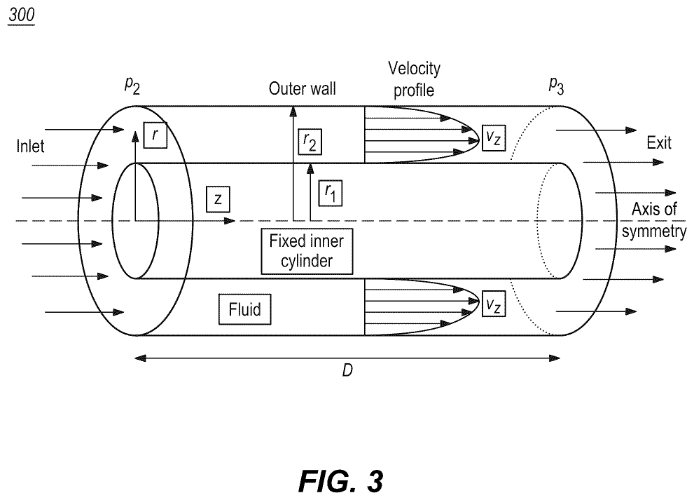

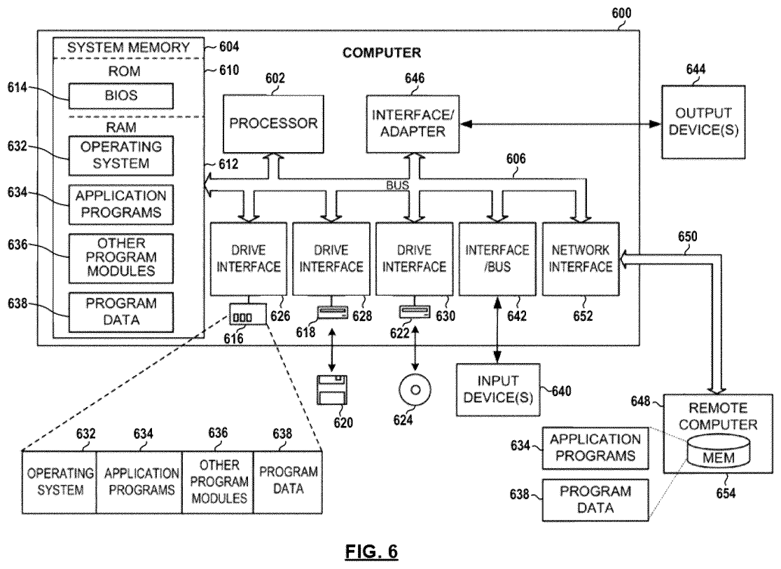

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 6. Thus, reference can be made to one or more examples of FIGS. 1-5 in the example of FIG. 6.

In this regard, FIG. 6 illustrates one example of a computer system 600 that can be employed to execute one or more embodiments of the present disclosure. Computer system 600 can be implemented on one or more general purpose networked computer systems, embedded computer systems, routers, switches, server devices, client devices, various intermediate devices/nodes or standalone computer systems. Additionally, computer system 600 can be implemented on various mobile clients such as, for example, a personal digital assistant (PDA), laptop computer, pager, and the like, provided it includes sufficient processing capabilities.

Computer system 600 includes processing unit 602, system memory 604, and system bus 606 that couples various system components, including the system memory 604, to processing unit 602. Dual microprocessors and other multi-processor architectures also can be used as processing unit 602. System bus 606 may be any of several types of bus structure including a memory bus or memory controller, a peripheral bus, and a local bus using any of a variety of bus architectures. System memory 604 includes read only memory (ROM) 610 and random access memory (RAM) 612. A basic input/output system (BIOS) 614 can reside in ROM 612 containing the basic routines that help to transfer information among elements within computer system 600.

Computer system 600 can include a hard disk drive 616, magnetic disk drive 618, e.g., to read from or write to removable disk 620, and an optical disk drive 622, e.g., for reading CD-ROM disk 624 or to read from or write to other optical media. Hard disk drive 616, magnetic disk drive 618, and optical disk drive 622 are connected to system bus 606 by a hard disk drive interface 626, a magnetic disk drive interface 628, and an optical drive interface 630, respectively. The drives and associated computer-readable media provide nonvolatile storage of data, data structures, and computer-executable instructions for computer system 600. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and disclosed herein. A number of program modules may be stored in drives and RAM 610, including operating system 632, one or more application programs 634, other program modules 636, and program data 638. In some examples, the application programs 634 can include one or more modules (or block diagrams), or systems, as shown and disclosed herein. Thus, in some examples, the application programs 634 can include system 100 or rheology analyzer 140.

A user may enter commands and information into computer system 600 through one or more input devices 640, such as a pointing device (e.g., a mouse, touch screen), keyboard, microphone, joystick, game pad, scanner, and the like. These and other input devices are often connected to processing unit 602 through a corresponding port interface 642 that is coupled to the system bus, but may be connected by other interfaces, such as a parallel port, serial port, or universal serial bus (USB). One or more output devices 644 (e.g., display, a monitor, printer, projector, or other type of displaying device) is also connected to system bus 606 via interface 646, such as a video adapter.

Computer system 600 may operate in a networked environment using logical connections to one or more remote computers, such as remote computer 648. Remote computer 648 may be a workstation, computer system, router, peer device, or other common network node, and typically includes many or all the elements described relative to computer system 600. The logical connections, schematically indicated at 650, can include a local area network (LAN) and a wide area network (WAN). When used in a LAN networking environment, computer system 600 can be connected to the local network through a network interface or adapter 652. When used in a WAN networking environment, computer system 600 can include a modem, or can be connected to a communications server on the LAN. The modem, which may be internal or external, can be connected to system bus 606 via an appropriate port interface. In a networked environment, application programs 634 or program data 638 depicted relative to computer system 600, or portions thereof, may be stored in a remote memory storage device 654.

Although this disclosure includes a detailed description on a computing platform and/or computer, implementation of the teachings recited herein are not limited to only such computing platforms. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models (e.g., software as a service (Saas, platform as a service (PaaS), and/or infrastructure as a service (IaaS)) and at least four deployment models (e.g., private cloud, community cloud, public cloud, and/or hybrid cloud). A cloud computing environment can be service oriented with a focus on statelessness, low coupling, modularity, and semantic interoperability.

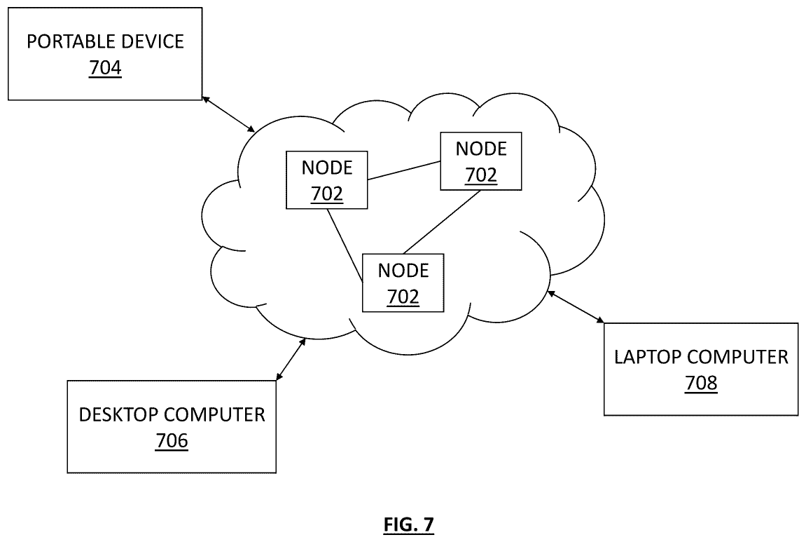

FIG. 7 is an example of a cloud computing environment 700 that can be used for implementing one or more modules and/or systems in accordance with one or more examples, as disclosed herein. Thus, reference can be made to one or more examples of FIGS. 1-6 in the example of FIG. 7. As shown, cloud computing environment 700 can include one or more cloud computing nodes 702 with which local computing devices used by cloud consumers (or users), such as, for example, personal digital assistant (PDA), cellular, or portable device 704, a desktop computer 706, and/or a laptop computer 708, may communicate. The computing nodes 702 can communicate with one another. In some examples, the computing nodes 702 can be grouped (not shown) physically or virtually, in one or more networks, such as Private, Community, Public, or Hybrid clouds, or a combination thereof. This allows the cloud computing environment 700 to offer infrastructure, platforms and/or software as services for which a cloud consumer does not need to maintain resources on a local computing device. The devices 704-708, as shown in FIG. 7, are intended to be illustrative and computing nodes 702 and cloud computing environment 700 can communicate with any type of computerized device over any type of network and/or network addressable connection (e.g., using a web browser). In some examples, the one or more computing nodes 702 are used for implementing one or more examples disclosed herein relating to root-source identification. Thus, in some examples, the one or more computing nodes can be used to implement modules, platforms, and/or systems, as disclosed herein.

In some examples, the cloud computing environment 700 can provide one or more functional abstraction layers. It is to be understood that the cloud computing environment 700 need not provide all of the one or more functional abstraction layers (and corresponding functions and/or components), as disclosed herein. For example, the cloud computing environment 700 can provide a hardware and software layer that can include hardware and software components. Examples of hardware components include: mainframes; RISC (Reduced Instruction Set Computer) architecture based servers; servers; blade servers; storage devices; and networks and networking components. In some embodiments, software components include network application server software and database software.

In some examples, the cloud computing environment 700 can provide a virtualization layer that provides an abstraction layer from which the following examples of virtual entities may be provided: virtual servers; virtual storage; virtual networks, including virtual private networks; virtual applications and operating systems; and virtual clients. In some examples, the cloud computing environment 700 can provide a management layer that can provide the functions described below. For example, the management layer can provide resource provisioning that can provide dynamic procurement of computing resources and other resources that are utilized to perform tasks within the cloud computing environment. The management layer can also provide metering and pricing to provide cost tracking as resources are utilized within the cloud computing environment 700, and billing or invoicing for consumption of these resources. In one example, these resources may include application software licenses. Security provides identity verification for cloud consumers and tasks, as well as protection for data and other resources. The management layer can also provide a user portal that provides access to the cloud computing environment 700 for consumers and system administrators. The management layer can also provide service level management, which can provide cloud computing resource allocation and management such that required service levels are met. Service Level Agreement (SLA) planning and fulfillment can also be provided to provide pre-arrangement for, and procurement of, cloud computing resources for which a future requirement is anticipated in accordance with an SLA.

In some examples, the cloud computing environment 700 can provide a workloads layer that provides examples of functionality for which the cloud computing environment 700 may be utilized. Examples of workloads and functions which may be provided from this layer include: mapping and navigation; software development and lifecycle management; virtual classroom education delivery; data analytics processing; and transaction processing. Various embodiments of the present disclosure can utilize the cloud computing environment 700.

The present disclosure is also directed to the following exemplary embodiments, which can be practiced in any combination thereof:
Embodiment 1: A combination of claims 1-7.
Embodiment 2: A combination of claims 8-12.
Embodiment 3: A combination of claims 13-17.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware based systems that perform the specified functions or acts or carry out combinations of special purpose hardware and computer instructions.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof. In addition, the use of ordinal numbers (e.g., first, second, third, etc.) is for distinction and not counting. For example, the use of “third” does not imply there must be a corresponding “first” or “second.” Also, as used herein, the terms “coupled” or “coupled to” or “connected” or “connected to” or “attached” or “attached to” may indicate establishing either a direct or indirect connection, and is not limited to either unless expressly referenced as such. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The term “based on” means “based at least in part on.” The terms “about” and “approximately” can be used to include any numerical value that can vary without changing the basic function of that value. When used with a range, “about” and “approximately” also disclose the range defined by the absolute values of the two endpoints, e.g. “about 2 to about 4” also discloses the range “from 2 to 4.” Generally, the terms “about” and “approximately” may refer to plus or minus 5-10% of the indicated number.

What has been described above include mere examples of systems, computer program products and computer-implemented methods. It is, of course, not possible to describe every conceivable combination of components, products and/or computer-implemented methods for purposes of describing this disclosure, but one of ordinary skill in the art can recognize that many further combinations and permutations of this disclosure are possible. The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

1. A system for measuring the rheology of drilling fluid used in a borehole having an annulus, the system comprising:
one or more microchip sensors configured to traverse at least a portion of the annulus along with the drilling fluid to thereby measure pressure and temperature as functions of depth or time in said portion; and
a rheology analyzer comprising a computing platform, including a processor and a memory, the rheology analyzer configured to implement a viscosity mapper, the viscosity mapper operable to receive said pressure and temperature measurements and apply a modified Poiseuille's formula to determine a mapping of rheological properties of the drilling fluid.

2. The system of claim 1 wherein the viscosity mapper is further configured to receive one or more of drilling fluid flow rate, equivalent shear, annulus radius and distance between different microchips to apply to the modified Poiseuille's formula.

3. The system of claim 1 wherein the modified Poiseuille's formula comprises:
$$𝒬 = \frac{π ( {r}_{2}^{2} - {r}_{1}^{2} )}{8 μ} ( - \frac{\partial p}{\partial z} ) [ {r}_{2}^{2} + {r}_{1}^{2} - \frac{{r}_{2}^{2} - {r}_{1}^{2}}{ln ( {r}_{2} / {r}_{1} )} ]$$
where Q is flow rate, r₁ is annulus radius, r₂ is borehole radius, μ is viscosity of fluid, and p is pressure.

4. The system of claim 1 wherein the one or more microchip sensors each comprise at least one magnetometer to detect a position of each of the plurality of microchip sensors against the depth of the annulus.

5. The system of claim 1 wherein the one or more microchip sensors each comprise at least one accelerometer to detect a position of each of the plurality of microchip sensors against the depth of the annulus.

6. The system of claim 1 wherein the mapping of rheological properties of the drilling fluid further comprises fluid flow speed and viscosity of the drilling fluid inside the annulus.

7. The system of claim 1 wherein the plurality of microchip sensors further comprise a recovery mechanism for receiving the said pressure and temperature measurements from the microchip sensors for delivery to the viscosity mapper.

8. A method for measuring drilling fluid rheology of an annulus of a borehole, the method comprising:
delivering one or more microchip sensors along with the drilling fluid into the annulus;
obtaining from the delivered microchip sensors measurements of pressure and temperature as functions of depth or time in the annulus; and
applying a modified Poiseuille's formula to said measurements to determine a mapping of rheological properties of the drilling fluid.

9. The method of claim 8 further comprising receiving one or more of drilling fluid flow rate, equivalent shear, annulus radius and distance between different microchips to apply to the modified Poiseuille's formula

10. The method of claim 8 wherein the modified Poiseuille's formula comprises:
$$𝒬 = \frac{π ( {r}_{2}^{2} - {r}_{1}^{2} )}{8 μ} ( - \frac{\partial p}{\partial z} ) [ {r}_{2}^{2} + {r}_{1}^{2} - \frac{{r}_{2}^{2} - {r}_{1}^{2}}{ln ( {r}_{2} / {r}_{1} )} ]$$
where Q is flow rate, r1 is annulus radius, r2 is borehole radius, μ is viscosity of fluid, and p is pressure.

11. The method of claim 8 wherein delivering one or more microchip sensors along with the drilling fluid into the annulus occurs continuously during an in-situ drilling process to provide a semi-continuous data stream from the annulus.

12. The method of claim 8 further comprising recovering the one or more microchip sensors for receiving the said pressure and temperature measurements for delivery.

13. A computer-readable storage medium containing instructions for measuring drilling fluid rheology of an annulus of a borehole, wherein the instructions, when executed by a processor, cause the processor to perform operations comprising:
delivering one or more microchip sensors along with the drilling fluid into the annulus;
obtaining from the delivered microchip sensors measurements of pressure and temperature as functions of depth or time in the annulus; and
applying a modified Poiseuille's formula to said measurements to determine a mapping of rheological properties of the drilling fluid.

14. The computer-readable storage medium of claim 13, the set of instructions further causing the machine to perform the steps of receiving one or more of drilling fluid flow rate, equivalent shear, annulus radius and distance between different microchips to apply to the modified Poiseuille's formula

15. The computer-readable storage medium of claim 13 wherein the modified Poiseuille's formula comprises:
$$𝒬 = \frac{π ( {r}_{2}^{2} - {r}_{1}^{2} )}{8 μ} ( - \frac{\partial p}{\partial z} ) [ {r}_{2}^{2} + {r}_{1}^{2} - \frac{{r}_{2}^{2} - {r}_{1}^{2}}{ln ( {r}_{2} / {r}_{1} )} ]$$
where Q is flow rate, r1 is annulus radius, r2 is borehole radius, μ is viscosity of fluid, and p is pressure.

16. The computer-readable storage medium of claim 13 wherein delivering one or more microchip sensors along with the drilling fluid into the annulus occurs continuously during an in-situ drilling process to provide a semi-continuous data stream from the annulus.

17. The computer-readable storage medium of claim 13, the set of instructions further causing the machine to perform the steps of recovering the one or more microchip sensors for receiving the said pressure and temperature measurements for delivery.
